(19) United States
(12) Patent Application Publication
(10) Pub. No.: US 2009/0171226 A1
(43) Pub. Date: Jul. 2, 2009

(54) SYSTEM AND METHOD FOR EVALUATING VARIATION IN THE TIMING OF PHYSIOLOGICAL EVENTS

(75) Inventors: Shannon E. Campbell, Boulder, CO (US); Steven E. Pav, San Francisco, CA (US); Michael P. O'Neil, Danville, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/340,981

(22) Filed: Dec. 22, 2008

Related U.S. Application Data
(60) Provisional application No. 61/009,678, filed on Dec. 31, 2007.

(51) Int. Cl. A61B 5/04

(52) U.S. Cl. 600/508; 600/300

(57) ABSTRACT

In embodiments, methods and systems are provided for the calculation of one or more indices representing variability in the timing of events in a signal representing a physiological parameter. In embodiments, the method and system may utilize an infinite impulse response formulation for the calculation of the indices to minimize memory and computational overhead, while additionally making the indices more responsive to newer measurements.

### RELATED APPLICATION

This application claims priority from U.S. Provisional Application No. 61/009,678, fled, Dec. 31, 2007, which is hereby incorporated by reference herein in its entirety.

### BACKGROUND

The present disclosure relates generally to a method and a system for measuring the variability in timing of physiological events. Specifically, the disclosed techniques may be used to determine an index representing heart rate variability from the output of a pulse oximeter, generally using minimal memory and computational overhead.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Heart rate may depend on a balance between two different branches of the autonomic nervous system. One branch, the sympathetic nervous system, controls the “fight or flight response” and tends to accelerate heart rate. This may be offset by the parasympathetic nervous system, which controls the “rest and digest” functions and tends to lower heart rate. In a healthy person these two branches of the autonomic nervous system work in tandem to balance the heart rate. For this reason, in a healthy person the heart rate may have significant variability as minor changes affect each branch. This variability may be termed heart rate variability, or HRV, and may be measured by the variation of the beat-to-beat intervals over time.

However, in patients that have had a heart attack, significant heart disease, or other medical conditions, the HRV often decreases. This more stable heart rate may be correlated with the risk of mortality of the patient. For example, low HRV has been correlated with cardiac mortality in patients that have had heart attacks.

One technique for the measurement of HRV is to measure the interbeat distance of the largest peak, or R wave, of the data output from an electrocardiogram (ECG). The ECG data is often analyzed by using Fourier transform techniques to convert the time domain data to frequency domain data. The frequency domain data of the heart rate variability may be characterized by the presence of three major components: a high frequency component, a low frequency component and a very low frequency component. Each of the major frequency components normally associated with HRV has been found to correlate with a different physiological parameter of heart rate control, For example, the high frequency component is believed to represent control of the heart rate by the parasympathetic nervous system and may be related to respiration. The low frequency component is believed to be associated with both sympathetic and parasympathetic modulation of the heart rate. The very low frequency component remains more difficult to analyze, although studies have indicated a possible relationship with various long-term bodily functions such as thermoregulation or kidney function.

In addition to the major components of the frequency domain data, discussed above, one further important frequency component of heart rate variability has been found in even longer assessments than used for the very low frequency component, for example, over 24 hour periods. This ultra low frequency heart rate variability is only poorly understood, but may be a powerful risk indicator in predicting mortality in cardiovascular disease.

While ECG data may produce an accurate measurement of the heart rate, it has a number of problems that may make it difficult for common use. For example, an ECG requires conductive electrodes be placed in direct contact with a patient's skin. Furthermore, ECG units are often complex, expensive and non-portable. Frequency analysis techniques may place further restrictions on the use of HRV studies, since the collection of time domain data over long periods of time, with regular calculation of a Fourier transform, may require levels of memory and computing power not found in a portable data collection device.

### SUMMARY

Certain aspects commensurate in scope with the disclosure are set forth below. It should be understood that these aspects are presented merely to provide the reader with a brief summary of certain forms the disclosure might take and that these aspects are not intended to limit the scope of the disclosure. Indeed, the disclosure may encompass a variety of aspects that may not be set forth below.

An embodiment provides a method of evaluating the variation in the timing of physiological parameter. The method may include collecting physiological parameter data comprising a sequence of numerical values for the physiological parameter over time. One or more sums may be accumulated from the physiological parameter data and a running sample variance may be calculated from the sums. An index may be calculated from the running sample variance, which may provide an indication of the timing of the physiological parameter.

Another embodiment provides a method of evaluating the variation in the timing of a physiological parameter. The method may include collecting physiological parameter data comprising a sequence of numerical values for the physiological parameter over time. A sample interval separating two or more events in the physiological parameter data may be determined. The sample interval may be compared to a target interval, and a probability coefficient may be incremented if the sample interval is within a preset range of the sample interval. A running index may be calculated from the probability coefficient. The running index may provide an indication of the timing of the physiological parameter.

In another embodiment, a medical device is provided. The medical device may have a sensor configured to collect physiological parameter data comprising a sequence of numerical values for a physiological parameter over a time period. The medical device may also include a processor configured to process the physiological parameter data and a memory configured to store computer readable instructions. The contents of the memory may include computer readable instructions configured to direct the processor to collect the physiological parameter data from the sensor. The memory may also include computer readable instructions that may be configured to direct the processor to accumulate one or more sums from the physiological parameter data and calculate a running sample variance from the sums. Finally, the memory may include computer readable instructions that direct the processor to calculate an index from the running sample variance and provide an indication of the timing of the physiological parameter from the index.

In another embodiment, a medical device is provided. The medical device may have a sensor configured to collect physiological parameter data comprising a sequence of numerical values for a physiological parameter over a time period. The medical device may also include a processor configured to process the physiological parameter data and a memory configured to store programs. The contents of the memory may include computer readable instructions configured to direct the processor to collect the physiological parameter data from the sensor. The memory may also include computer readable instructions that may be configured to direct the processor to determine a sample interval separating two or more events in the physiological parameter data and compare the sample interval to a target interval. If the sample interval is within a preset range of the target interval, the computer readable instructions may be configured to direct the processor to increment a probability coefficient. Finally, the memory may include computer readable instructions to direct the processor to calculate a running index from the probability coefficient and provide an indication of the timing of the physiological parameter from the running index.

Another embodiment provides a tangible machine readable media that may include code for collecting physiological parameter data comprising a sequence of numerical values for a physiological parameter over time and code for accumulating one or more sums from the signal. The tangible machine readable media may also include code for calculating a running sample variance from the one or more sums, code for calculating an index from the running sample variance, and code for providing an indication of the timing of the physiological parameter based on the index.

Another embodiment provides a tangible machine readable media that may include code for collecting physiological parameter data signal comprising a sequence of numerical values for a physiological parameter over time and code for determining a sample interval separating two or more events in the signal. The tangible machine readable media may also include code for comparing the sample interval to a target interval, and incrementing a probability coefficient if the sample interval is within a preset range of the target interval. Finally, the tangible machine readable media may include code for calculating a running index from the probability coefficient and code for providing an indication of the variation in the timing of the physiological parameter based on the running index.

### BRIEF DESCRIPTION OF THE DRAWINGS

Advantages of the disclosure may become apparent upon reading the following detailed description and upon reference to the drawings in which:

FIG. 1 is a block diagram of a system for the measurement of a physiological parameter in accordance with an embodiment;

FIG. 2 is a flow chart showing a method for use in calculating a heart rate variability index in accordance with an embodiment;

FIG. 3 is a flow chart showing a method for calculating one or more indices reflecting heart rate variation in accordance with an embodiment;

FIG. 4 is a flow chart showing a method for calculating one or more indices reflecting heart rate variation in accordance with an embodiment;

FIG. 5 is a graphical representation of a heart rate sampled over about 24 hours;

FIG. 6 is a graphical representation of an IIR timescale exponent calculated from the heart rate of FIG. 5 in accordance with an embodiment; and

FIG. 7 is a graphical representation of an IIR uncertainty calculated from the heart rate of FIG. 5 in accordance with an embodiment.

### DETAILED DESCRIPTION

One or more embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Medical devices may be used to obtain signals representing physiological parameters from patients. However, these signals, which are sequences of numerical values over time, may have too much information or noise to be effectively used in the diagnosis or treatment of certain medical conditions, such as heart problems. Accordingly, the signals may be analyzed to generate a secondary series of numerical values, for example, an index representing heart rate variability, which may provide a more useful diagnostic tool for the medical condition. However, the calculation of a secondary series may be computationally intensive or otherwise difficult to implement.

Embodiments of the present disclosure provide a method that may be used to collect and analyze time domain data to generate an index representing the time variability of the signal. The method may use relatively inexpensive equipment and does not need complex calculations, such as a Fourier transform, for implementation. The method may be implemented on a pulse oximeter, or other types of portable units, for the long-term collection and analysis of heart rate variability data while a patient goes about his or her normal activities. However, the method described below is not limited to heart rate or pulse oximetry and may be implemented on other systems to calculate indices reflective of the variability of signals representing other physiological conditions. The analysis may be performed in real time or may be performed on a previously collected data set.

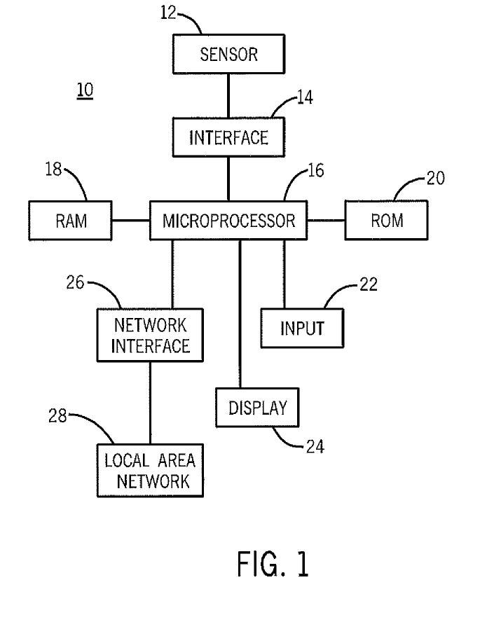

FIG. 1 is a block diagram of a medical device 10, which may be used in embodiments of the present disclosure. The medical device 10 may have a sensor 12 for the detection of a signal representing a physiological parameter. In an embodiment, the sensor 12 may be an optical sensor used with a pulse oximeter for the measurement of oxygen saturation in the bloodstream. However, the disclosed methods are not limited to pulse oximetry. For example, the sensor 12 may include electrodes for detecting signals from the heart, brain, or other organs. The signal from the sensor 12 may be conditioned by an interface 14 prior to being utilized by a microprocessor 16.

In an embodiment, the microprocessor 16 may be connected to random access memory (RAM) 18 and/or read-only memory (ROM) 20. The RAM 18 may be used to store the signals from the sensor 12 and the results of calculations that the microprocessor 16 performs. The ROM 20 may contain code to direct the microprocessor 16 in collecting and processing the signal and may be considered a tangible machine readable media. Other tangible machine readable media may be used in other embodiments, including, for example, hard disk drives, floppy disk drives, pen drives, optical drives, or any other devices that may be used in the art to contain code.

The microprocessor 16 may be connected to an input device 22 which may be used for local entry of control and calculation parameters for the medical device 10. A display unit 24 may be connected to the microprocessor 16 to display the results the microprocessor 16 has generated from the signal representing the physiological parameter.

The microprocessor 16 may also be connected to a network interface 26 for the transfer of data from the microprocessor 16 to devices connected to a local area network 28. The transferred data may, for example, include signal data, indices representing the status of physiological conditions, alarm signals, or any combination thereof. The transferred data may also include control signals from the devices on the local area network 28, for example, to instruct the medical device 10 to send signal data, or other information, to a device on the local area network 28.

In an embodiment the medical device 10 may be used to calculate an index representing heart rate variability (HRVI) with the data collected from the sensor 12, using the method discussed below. The HRVI may be output to the display unit 24 or sent to a network device on the local area network 28. The processing may take place in real time, or may be run after the data collection is completed for later determination of an HRVI.

In another embodiment, a network device located on the local area network 28 may be used to calculate an HRVI with the data collected from the sensor 12, using the method discussed below. In this embodiment, the network device may request that the signal be sent from the medical device 10 through the network interface 26. As for the embodiment discussed above, the network device may be used to either determine the HRVI in real time or to process a previously collected signal. Furthermore, the code that may be used to direct the network device to obtain and analyze the signal may be contained on a tangible machine readable media, as discussed above.

In either of the embodiments discussed above, the value of the HRVI may be used to trigger one or more alarms, alerting practitioners to clinically important conditions. These alarms may appear on devices on the local area network 28, for example, a patient monitoring screen in an intensive care unit. Alternatively, the alarms may appear on the display unit 24 of the medical device 10. Further, it may be advantageous to activate alarms in both locations using the results from either a local calculation on the medical device 10 or from a remote calculation on a network device connected to the local area network 28.

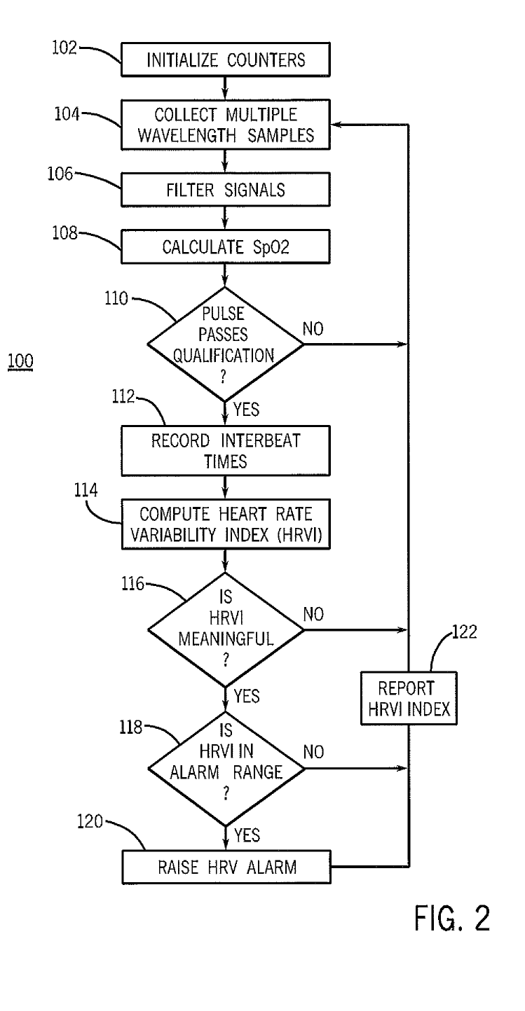

FIG. 2 is a flow chart showing an embodiment of a method 100 for use in calculating a heart rate variability index from data collected using a pulse oximeter. The method is not limited to a pulse oximeter, but may be implemented on other devices for the determination of indices corresponding to time variations in other signals representing physiological parameters. The method 100 begins by initializing the counters needed for the accumulation of summation data, used to calculate the heart rate variability index, as shown in block 102. One set of counters may be used for each time scale selected for monitoring. In an embodiment in which one or more indices are monitored in real time, the initialization may be performed when monitoring is first started. In other embodiments, for example, when the method may be implemented on a device connected to a local area network 28, as shown in FIG. 1, the initialization of the counters may be performed when either starting to monitor the physiological parameter in real time or starting the analysis of a previously collected data set.

After initialization of the counters, multiple wavelength samples may be collected as shown in block 104. The signals from the samples may be filtered, as shown in block 106, prior to being used to calculate a value for the SpO₂ in block 108. The SpO₂ signal may be analyzed to identify a pulse from a patient. In block 110, the pulse may be qualified to ensure that it is actually due to a signal from a heart beat and not from noise. In an embodiment, the acts described with respect to blocks 104-110 may be performed according to the techniques discussed in U.S. Pat. No. 5,853,364, herein incorporated by reference in its entirety for all purposes.

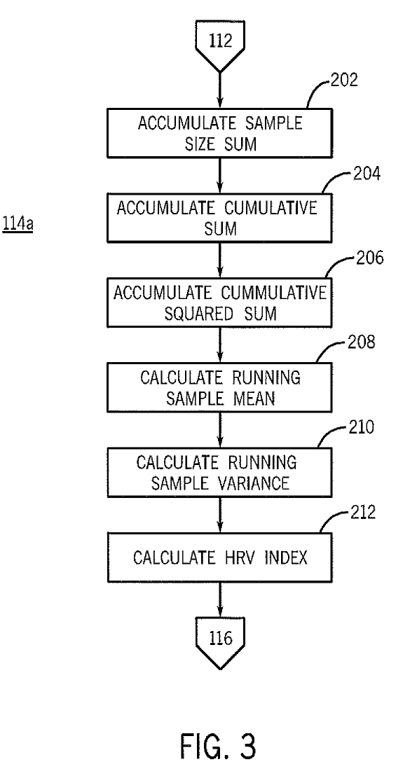

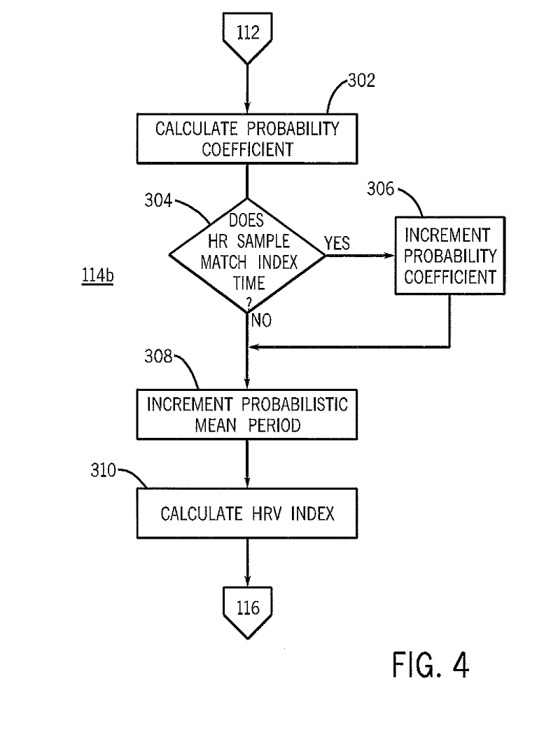

After the pulse is qualified, the inter-beat times may be recorded, as shown in block 112. In an embodiment, the inter-beat time may be determined by measuring the separation in time between the peak signals from a pulse oximetry plethysmogram obtained from a pulse oximeter. In block 114, a heart rate variability index (HRVI) is calculated. In an embodiment, the HRVI may be determined by the method detailed in FIG. 3. In another embodiment, the HRVI may be determined by the method detailed in FIG. 4. Once the HRVI has been determined, the method 100 may determine if enough samples have been collected to ensure that the HRVI is meaningful, as shown in block 116. If not enough samples have been collected, the method 100 may resume with the acts starting at block 104.

If an alarm range for the HRVI has been set, the HRVI may be compared to the alarm range, as shown in block 118. If the value is within the alarm range then the alarm may be activated, as shown in block 120. In either case, the HRVI may be reported to the user in block 122. The method then returns to block 104 to collect the next wavelength sample. In an embodiment, the HRVI may be output to a display 24 connected to the medical device 10. In another embodiment, the HRVI may be output using network interface device 26 and displayed on a device attached to a local area network 28.

FIG. 3 is a flow chart showing a method 114a for calculating one or more indices reflecting heart rate variation HRVI, in accordance with an embodiment. This may be considered a detailed view of a method that may be used in block 114 of FIG. 2. The index generated by this method may be termed the infinite impulse response (IIR) timescale exponent. When an embodiment using either the method 114a detailed in FIG. 3 or the method 114b detailed in FIG. 4 to monitor indices in real-time, the equations shown as summations below may actually represent the single value accumulated at the time the current sample is acquired. In other embodiments, such as when a previously acquired data set is analyzed, the summations may be calculated for the entire data set at the time of analysis.

In block 202 of FIG. 3, a sample size sum is accumulated. In an embodiment, this accumulation may be performed using the formula shown in equation 1:

$${n}_{m} ( r ) = {\sum }_{i = 0}^{m - 1} {r}_{1}^{i} \qquad equation 1$$

where r is a term that represents the “half-life” of memory in an infinite impulse response (IIR) algorithm. The value of r is calculated as the negative of log(2) divided by log(r₁). In calculating r, r₁ may be selected to enhance the sensitivity of the index to more recently collected data, For example, if multiple values of the index are calculated at different values of r, the power over the different timescales can be estimated. For example, in an embodiment, r₁ may be selected to be 0.99999198, which corresponds to a half life of around 24 hours, assuming a mean heart rate of around 60 beats-per-minute. In another embodiment, r₁ may be selected to be 0.9977, which corresponds to a half life of around 5 minutes.

A cumulative sum may be accumulated, as shown in block 204. In an embodiment, this accumulation may be performed using the formula shown in equation 2:

$${s}_{1 , m}^{( X )} ( r ) = {\sum }_{i = 0}^{m - 1} {r}^{i} {X}_{m - i} \qquad equation 2$$

where rⁱ is the half life term, discussed above, and Xₘ₋ᵢ is the last value of the inter-beat separation, as calculated from the pulse oximetry data.

A cumulative squared sum may be accumulated, as shown in block 206. In an embodiment, this accumulation may be performed using the formula shown in equation 3:

$${s}_{2 , m}^{( X )} ( r ) = {\sum }_{i = 0}^{m - 1} {r}^{i} {X}_{m - i}^{2} \qquad equation 3$$

where r is the half life term discussed above and X²ₘ₋₁ is the last value measured for the inter-beat separation. After each set of sums is accumulated, the sums may be used to calculate the heart rate variability index.

The sums accumulated above may be used to calculate a running sample mean, as shown in block 208. In an embodiment, the running sample mean may be calculated using the formula given in equation 4:

μₘ^{(X)}(r)=s_{1,m}^{(X)}(r)/nₘ(r) equation 4

where s_{1,m}^{(X)} is the cumulative sum, as calculated in block 204, and nₘ(r) is the sample size sum, as calculated in block 202. The use of the IIR weighting factor, r, in the calculation of the sums, weighs more recent values for the inter-beat time more heavily than older values, and may help the HRVI to reflect current changes in the heart rate.

A running sample variance may be calculated, as shown in block 210. In an embodiment, the running sample variance may be calculated using the formula given in equation 5:

$${σ}_{m}^{( X )} ( r ) = \sqrt{\frac{{s}_{2 , m}^{( X )} - {μ}_{m}^{( X )} ( r ) {s}_{1 , m}^{( X )} ( r )}{{n}_{m} ( r ) - 1}} \qquad equation 5$$

From the running sample mean, calculated in block 208, and the running sample variance, calculated in block 210, the HRVI may be calculated in block 212. For example, the HRVI for each timescale may be calculated by determining the best fit slope of the log-linear regression of the running sample variance to the timescale. In an embodiment, this may be performed by fitting the function

$${\{ σ \frac{( x )}{m} ( {r}_{k} ) \}}_{k = I}$$

to the l values used for the timescale (r).

In another embodiment, the HRVI may be determined based on a probabilistic calculation of the uncertainty in the signal, as discussed below for FIG. 4. FIG. 4 is a flow chart showing a method 114b for calculating one or more indices reflecting heart rate variation, in accordance with an embodiment. This figure represents a detailed view of a method 114b that may be used in block 114 of FIG. 2 to calculate HRVI. The index calculated in this embodiment may be termed the IIR uncertainty. As shown in block 302, a probability coefficient, qⱼ, may be calculated by setting the value of qⱼ equal to r times the current value of q, where r represents an IIR weighting factor between zero and one. The use of the IIR weighting factor in the calculations weights more recent values for the inter-beat time more heavily than older values, and, thus, may help the HRVI to continue to reflect current changes in the heart rate.

The inter-beat time sample may be compared to an index time previously selected, as shown in block 304. If there is a match between the inter-beat time and the index time, then in block 306 the probability coefficient, qⱼ, may be incremented by one. Further, a range may be used around the index time. Thus, in an embodiment, if an inter-beat time lands within the range, qⱼ may be incremented by one.

In block 308, a probabilistic mean period \tilde{m} may be calculated by setting the value for equal to one plus (r times the current value of \tilde{m}). In block 310, the probabilistic mean period may be used to calculate HRVI. In an embodiment, the HRVI may be calculated using the formula shown in equation 7:

$${H}_{i} ← {log}_{2} \tilde{m} - \frac{1}{\tilde{m}} ( {\sum }_{j = 1}^{n} = {log}_{2} {q}_{j} ) \qquad equation 7$$

where Hᵢ is the HRVI, \tilde{m} is the probabilistic mean period, and qⱼ is the probability coefficient calculated in block 302.

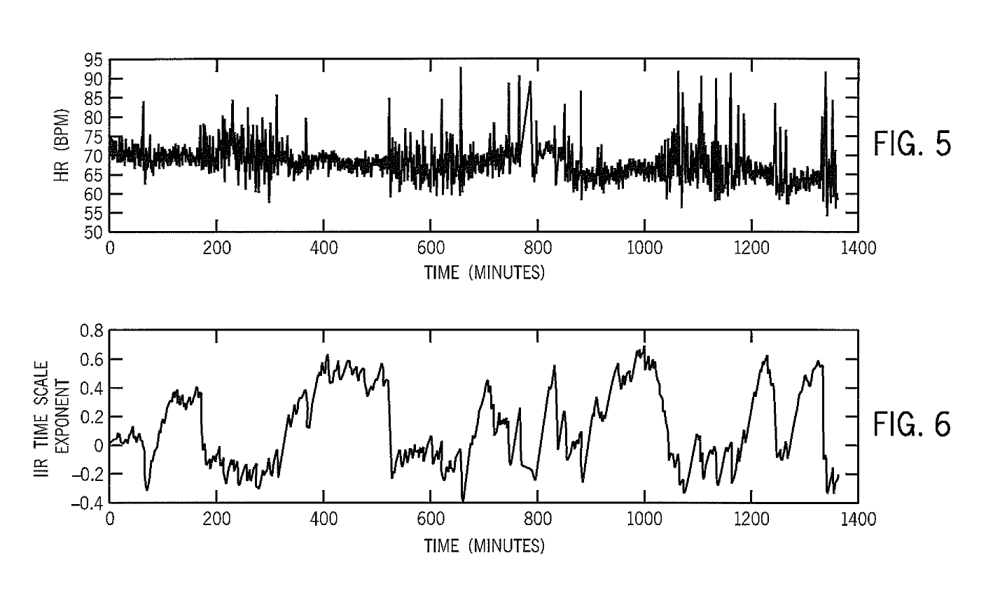

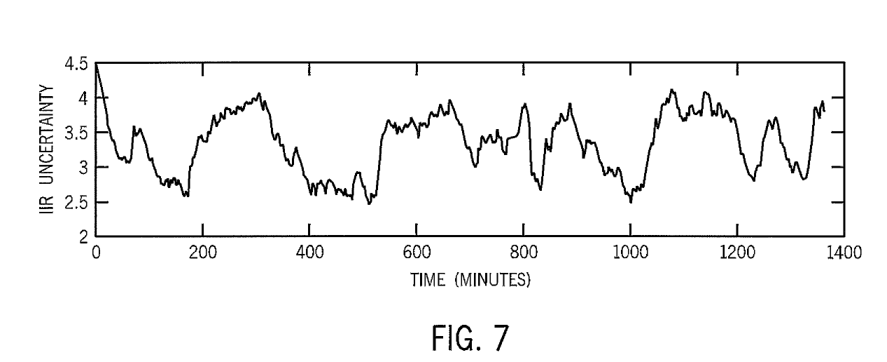

The operation of the embodiments discussed above may be illustrated by the charts in FIGS. 5, 6, and 7. FIG. 5 is a chart of a heart rate, on the vertical axis, sampled over a nearly 24 hour period, and charted against the time, in minutes, on the horizontal axis. FIG. 6 is a chart of the IIR timescale exponent calculated from the heart rate of FIG. 5 using the embodiment discussed with respect to FIG. 3. The vertical axis for FIG. 6 is expressed in relative units related to the calculated value, while the horizontal axis is time, in minutes. FIG. 7 is chart of the IIR uncertainty calculated from the heart rate of FIG. 5 using the embodiment discussed with respect to FIG. 4. The vertical axis for FIG. 7 is expressed in relative units related to the calculated value, while the horizontal axis is time, in minutes.

In both FIGS. 6 and 7 an IIR factor was selected to correspond to a half life of approximately 9.47 minutes. From these charts, it can be noted that the two indices are roughly complimentary, for example, the IIR timescale exponent increases and the IIR uncertainty decreases during periods when the short-term variation is less than the historical variation. Either index may be useful for quantifying heart rate in comparison to pre-selected timeframes for inter-beat separation. In one embodiment, periods of low heart variability, as shown by a high value for the IIR timescale exponent or a low value for the IIR uncertainty, may indicate that the patient should be more closely monitored for problematic conditions.

In another embodiment, the HRVI may be useful for the diagnosis of obstructive sleep apnea from the heart rate variability. In this embodiment, an HRVI may be calculated using the method above and an IIR factor giving a half life of between about 30 to 70 seconds. A high value for the IIR timescale exponent in this range or a corresponding low value for the IIR uncertainty may indicate the presence of obstructive sleep apnea.

While the disclosure may be susceptible to various modifications and alternative forms, specific embodiments have been shown by way of example in the drawings and have been described in detail herein. However, it should be understood that the disclosure is not intended to be limited to calculating an index representing heart rate variability. Indeed, the present techniques may not only be applied to heart rate variability indices, but may also be utilized for the analysis of the time separation of other physiological events. Rather, the disclosure is to cover all modifications, equivalents, and alternatives falling within the spirit and scope of the disclosure as defined by the following appended claims.

What is claimed is:

1. A method of evaluating variation in the timing of a physiological parameter, comprising:
collecting physiological parameter data comprising a sequence of numerical values for the physiological parameter over time;
accumulating one or more sums from the physiological parameter data;
calculating a running sample variance based at least in part upon the one or more sums;
calculating an index based at least in part upon the running sample variance; and
providing an indication of timing of the physiological parameter based at least in part upon the index.

2. The method of claim 1, wherein the one or more sums comprise a sample size sum, a cumulative sum, and/or a cumulative squared sum.

3. The method of claim 1, comprising calculating a running sample mean from the one or more sums.

4. The method of claim 1, wherein the physiological parameter comprises a heart rate.

5. A medical device, comprising:
a sensor capable of collecting physiological parameter data, the physiological parameter data comprising a sequence of numerical values for a physiological parameter over a time period;
a processor capable of processing the physiological parameter data; and
a memory capable of storing computer readable instructions, wherein the contents of the memory comprises computer readable instructions capable of directing the microprocessor to:
collect the physiological parameter data from the sensor;
accumulate one or more sums from the physiological parameter data;
calculate a running sample variance based at least in part upon the one or more sums;
calculate an index based at least in part upon the running sample variance; and
provide an indication of the timing of the physiological parameter based at least in part upon the index.

6. The medical device of claim 5, comprising a pulse oximeter.

7. The medical device of claim 5, wherein the physiological parameter comprises a heart rate.

8. The medical device of claim 5, wherein the one or more sums comprise a sample size sum, a cumulative sum, and/or a cumulative squared sum.

9. The medical device of claim 5, wherein the contents of the memory comprises computer readable instructions capable of directing the microprocessor to calculate a running sample mean from the one or more sums.

10. A tangible machine readable media having instructions stored thereon, when, if executed cause a method to be performed, the method, comprising:
collecting physiological parameter data comprising a sequence of numerical values for a physiological parameter over time;
accumulating one or more sums from the physiological parameter data;
calculating a running sample variance based at least in part upon the one or more sums;
calculating an index based at least in part upon the running sample variance; and
providing an indication of the timing of the physiological parameter based at least in part upon the index.

11. The tangible machine readable media of claim 10, wherein the physiological parameter comprises a heart rate.

12. The tangible machine readable media of claim 10, wherein the one or more sums comprise a sample size sum, a cumulative sum, or a cumulative squared sum, and/or combinations thereof.

13. The tangible machine readable media of claim 10, further comprising instructions for calculating a running sample mean based at least in part upon the one or more sums.
